(19) United States
(12) Patent Application Publication
(10) Pub. No.: US 2025/0058135 A1
(43) Pub. Date: Feb. 20, 2025

(54) METHOD FOR CORRECTING A PERSON’S SLEEP PARAMETERS AND SYSTEM FOR THE IMPLEMENTATION THEREOF

(71) Applicant: "SLEEP AND WAKE NEUROTECHNOLOGY CENTER" LIMITED LIABILITY COMPANY, Moscow (RU)

(72) Inventor: Ilya Sergeevich BLOKHIN, Moscow (RU)

(21) Appl. No.: 18/724,798

(22) PCT Filed: Dec. 26, 2022
(86) PCT No.: PCT/RU2022/050412

§ 371 Date: Jun. 27, 2024

(30) Foreign Application Priority Data
Dec. 27, 2021 (RU) 2021139068

(51) Int. Cl. A61N 1/40; A61M 21/00; A61M 21/02

(52) U.S. Cl. CPC A61N 1/40; A61M 21/02; A61M 2021/0055

(57) ABSTRACT

This invention can be used to reduce the average latency of human sleep, improve sleep continuity, increase the amount of slow-wave sleep, and simplify awakening. The group of inventions allows correcting the human sleep parameters by generating short (“spike”) electric field pulses of no less than 20 milliseconds, preferably ranging from no less than 20 milliseconds to no more than 1000 milliseconds, near a sleeping person in accordance with the specified algorithm.

### TECHNICAL FIELD

This invention can be used to reduce the average latency of human sleep, improve sleep continuity, increase the amount of slow-wave sleep, and simplify awakening.

### PRIOR ART

Pulsed Electromagnetic Field (PEMF) technology is widely used around the world for therapeutic purposes, such as achieving the normalizing effect on human sleep.

Most PEMF-based inventions use rectangular-shaped pulses of the current applied to an inductor coil that generates a pulsed magnetic field [U.S. Pat. No. 8,808,159, Aug. 19, 2014; Germany, application Ser. No. 10/200,4002218, Aug. 18, 2005; Russian patent 2170109, Jul. 10, 2001]. In practice, the sinusoidal pulses are not used at all due to their proven low efficiency. However, the shape of the pulse would not have been so significant, if the normalizing effect was provided by the magnetic component of the field. This is confirmed by the fact that PEMF demonstrates extremely poor efficiency in normalizing sleep if it is implemented by means of sinusoidal pulses, where the change in the magnetic field value is “slow.” On a separate note, it should be mentioned that a constant magnetic field affects only moving charged particles, while an alternating magnetic field impacts the static particles. Since we are focusing on a particular biological effect, it is clear that a biological organism is dominated by charged particles in a bound state (as part of molecules or molecular groups) with a small number of degrees of freedom and motion amplitude.

### SUMMARY OF INVENTION

Therefore, the problem addressed by this invention is to abandon the magnetic component of the pulses, namely, the part of the pulse which generates a constant magnetic field (“plateau” of the pulse, when a constant current runs through the inductor coil) while preserving the overall efficiency of the method. In this case, the technical result achieved by addressing this problem involves improving the sleep normalization effect at all stages of the sleep while eliminating the discomfort experienced by people sensitive to magnetic fields during their prolonged stay near a pulsed magnetic field source.

To achieve this result, we propose the following method for correcting human sleep parameters:
generating short (“spike”) electric field pulses of no less than 20 milliseconds, preferably ranging from no less than 20 milliseconds to no more than 1000 milliseconds;
using generated pulses near the sleeping person according to at least one of the following algorithms (sequentially or any such algorithm(s) based on the existing needs):
improving sleep continuity as measured by the number of transitions from the second stage of the sleep cycle to the first stage and to the waking state;
reducing average sleep latency by gradually changing the duration of pulses over the first 20-30 minutes of sleep;
simplifying awakening process by gradually changing the duration of pulses over the last 30-60 minutes of sleep;
increasing the amount of slow sleep in the second and third stages of the sleep cycle.

The pulses can be generated by electromagnetic induction caused, in particular, by the rate of change in the value of the magnetic field produced by the magnetic field source, which is provided by a flat bifilar inductor coil, while the rate of change in the magnetic field value is controlled by the rate of rise of the leading edge of electric pulses fed to the coil, and such pulses have rectangular shape with a fill factor of less than 5%.

To achieve this result, we propose a system that implements the above method for correcting human sleep parameters and includes a unit for generating short (“spike”) pulses of electric field; a memory and control unit for the above pulse generation unit; a time control unit used to synchronize the generated “spike” pulses with the periods of falling asleep, deep sleep and/or awakening.

In the claimed system, the generation unit produces short (“spike”) pulses by electromagnetic induction and is designed to change the amplitude of short (“spike”) pulses through the rate of rise and amplitude of the leading edge of electric pulses fed to the time-varying magnetic field shaper; the said time-varying magnetic field shaper can be made in the form of a flat bifilar inductor coil designed to receive an amplified electric signal with the specified duration parameters of pulses, which have a rectangular shape with a fill factor of less than 5%, and the said electric signal with the specified duration parameters and fill factor is generated by the converter. In addition, the said algorithms are stored in a memory unit synchronized with the said time control unit to ensure that the generated “spike” pulses of electric field with a certain duration are aligned in time with periods of falling asleep, deep sleep and awakening, and the said control algorithms can be communicated to the said memory unit through a mobile app using the said Bluetooth module, or by a remote server through the said Wi-Fi module.

The capabilities of the claimed system also include such options as enabling the persons who need to adjust their sleep parameters to define the value of electromagnetic induction effect (power of “spike” electric field pulses) by selecting the distance to the said time-varying magnetic field shaper or by controlling the amplitude and rate of rise of the leading edge of electric pulses fed to the said shaper. In addition, the persons who need to adjust their sleep parameters can select an algorithm that allows to either adjust all of the said sleep parameters, or any of the said parameters individually.

In the claimed group of inventions, this result can be achieved by using the change of the magnetic field on the leading edge of the pulse, when the current on the inductor coil changes suddenly and generates a short (“spike”) pulse of electric field in all nearby closed conductive circuits as a result of EMF which, in turn, allows to reduce the pulse duty cycle to 1%, i.e., to practically abandon the constant magnetic field component, while the effectiveness of the result is ensured by the rate of rise of the leading edge of the electric pulse fed to the inductor coil that determines the rate of change of the magnetic field.

The additional benefits provided by the claimed group of inventions are ensured through the use of rectangular-shaped electric pulses with a fill factor of less than 5%, which enables to “cut off” the constant magnetic field component (since it is generated only when a direct current flows through the coil) and leave only “helpful” “spike” pulses of the electric field near the sleeping person.

### BRIEF DESCRIPTION OF THE DRAWINGS

FIG. 1 shows the flowchart for this invention.

FIG. 2 presents the “spike” pulses of the electric field generated by time-varying magnetic field in accordance with the claimed invention.

### DETAILED DESCRIPTION OF THE EMBODIMENTS

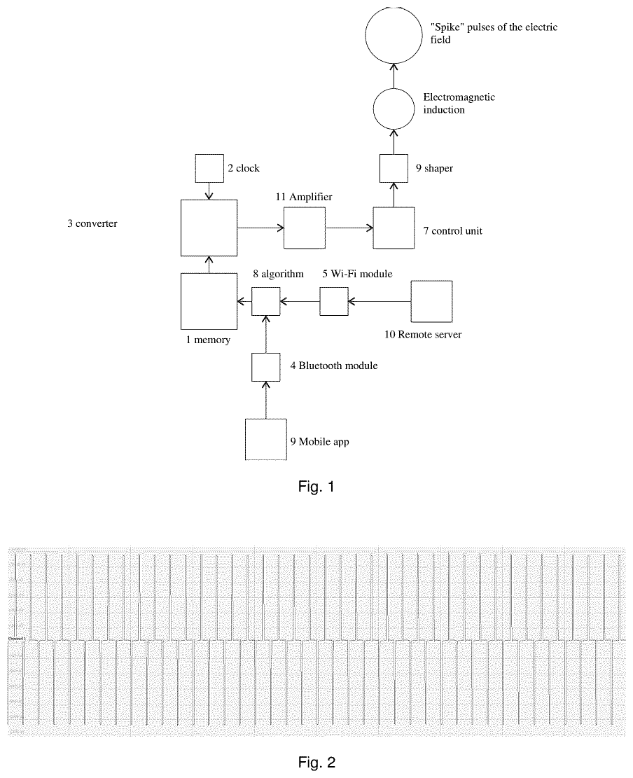

The claimed method can be implemented with the system described on the chart in FIG. 1.

The system that can adjust the human sleep parameters comprises a memory unit or “memory” 1, time control unit or “clock” 2, converter 3 that can use the control algorithm 8 to read the parameters (duration and fill factor) of the signal fed to the amplifier 11, which sends the amplified signal to the unit for generating short (“spike”) pulses of the electric field or time-varying magnetic field shaper 6. The control algorithm is recorded and stored in the memory unit 1 or communicated to it by Bluetooth module 4 through mobile app 9 or Wi-Fi module 5 from the remote server 10. The signal duration parameters are changed by synchronizing the start time and end time of the control algorithm with internal clock of the unit 2. The control unit for the rate of rise and amplitude of electric pulses 7 may be adjusted in such a way as to generate, near the sleeping person, the optimal parameters of “spike” electric field pulses responsible for

The provided algorithms are either stored in the memory 1 by the remote server 10 via Wi-Fi 5, or uploaded from the mobile app 9 via Bluetooth module 4, or selected from those already stored in the memory 1. The algorithm 8 starts to control the duration of the electric signal fed from the converter 3 to amplifier 11. The form of the electric signal pulses is fine-tuned by the control unit for the rate of rise and amplitude 7 initially configured by the users in accordance with the characteristics of their organism. The adjusted electric signal is fed to the time-varying magnetic field shaper 6, while the shaper itself is placed near the sleeping person at a distance that depends on the settings of the control unit used to control the rate of rise and amplitude of electric signals 7, and is determined by the users based on the characteristics of their organism.

According to Faraday's Law, an electromotive force (EMF) is generated with the onset of the leading edge of the electric signal pulse. Its value can be represented in vector form as follows:

$$rot \vec{E} = - \frac{\partial \overline{B}}{\partial t}$$

The derivative of the magnetic field B̅ is maximal at the maximum rate of rise of the leading edge of the pulse. Moreover, the value of rotĒ is affected not only and not so much by the value of the magnetic field induction B̅, but by the period of time ∂t required for its change.

This can be clearly seen, if an auxiliary inductor coil with an electric signal amplifier connected to its outputs is placed near the time-varying magnetic field shaper 6. The time-varying magnetic field will generate an EMF and induce current in the auxiliary coil through electromagnetic induction (“spike-like” electric pulses), as shown in FIG. 2.

A specific embodiment of the system's operation for reducing the average latency of human sleep is a “step change” in the frequency of “spike” pulses of the electric field near a sleeping person, as shown in Table 1, which provides an algorithm for changing the frequency of “spike” pulses of the electric field near a sleeping person to adjust (reduce) the average latency parameter of human sleep.

TABLE 1

| 1 | 2 | 3 | 4 | 5 | 6 | 7 | 8 | 9 | 10 |
|---|---|---|---|---|---|---|---|---|---|
| minute | minutes | minutes | minutes | minutes | minutes | minutes | minutes | minutes | minutes |
| 32 | 33 | 34 | 35 | 37 | 38 | 40 | 41 | 43 | 45 |
| milliseconds | milliseconds | milliseconds | milliseconds | milliseconds | milliseconds | milliseconds | milliseconds | milliseconds | milliseconds |
| 12 | 12 | 13 | 14 | 15 | 16 | 17 | 18 | 19 | 20 |
| minutes | minutes | minutes | minutes | minutes | minutes | minutes | minutes | minutes | minutes |
| 47 | 50 | 52 | 55 | 58 | 62 | 66 | 71 | 76 | 83 |
| milliseconds | milliseconds | milliseconds | milliseconds | milliseconds | milliseconds | milliseconds | milliseconds | milliseconds | milliseconds |
| 21 | 22 | 23 | 24 | 25 | 26 | 27 | 28 | 29 | 30 |
| minutes | minutes | minutes | minutes | minutes | minutes | minutes | minutes | minutes | minutes |
| 90 | 100 | 111 | 125 | 142 | 166 | 200 | 250 | 333 | 500 |
| milliseconds | milliseconds | milliseconds | milliseconds | milliseconds | milliseconds | milliseconds | milliseconds | milliseconds | milliseconds |

A specific embodiment of the system's operation for improving the sleep continuity estimated by the number of transitions from stage 2 to stage 1 of the sleep cycle and to the waking state is the generation of “spike” pulses of the electric field near a sleeping person for as long as 500 milliseconds from the time when the person falls asleep, as estimated by the onset of stage 3 of the sleep cycle and 60 minutes prior to the expected time of awakening.

A specific embodiment of the system's operation for increasing the amount of slow-wave sleep on stage 2 and stage 3 of the sleep cycle is the generation of “spike” pulses of the electric field near a sleeping person for as long as 500 milliseconds from the time when the person falls asleep, as estimated by the onset of stage 3 of the sleep cycle and 60 minutes prior to the expected time of awakening.

A specific embodiment of the system's operation for simplifying the awakening is a “step change” in the frequency of “spike” pulses of the electric field near a sleeping person, as shown in Table 2, which provides an algorithm for changing the frequency of “spike” pulses of the electric field near a sleeping person to simplify the awakening of such person.

TABLE 2

| 1 | 2 | 3 | 4 | 5 | 6 | 7 | 8 | 9 | 10 |
|---|---|---|---|---|---|---|---|---|---|
| minute | minutes | minutes | minutes | minutes | minutes | minutes | minutes | minutes | minutes |
| 500 | 333 | 250 | 200 | 166 | 142 | 125 | 111 | 100 | 90 |
| milliseconds | milliseconds | milliseconds | milliseconds | milliseconds | milliseconds | milliseconds | milliseconds | milliseconds | milliseconds |
| 12 | 12 | 13 | 14 | 15 | 16 | 17 | 18 | 19 | 20 |
| minutes | minutes | minutes | minutes | minutes | minutes | minutes | minutes | minutes | minutes |
| 83 | 76 | 71 | 66 | 62 | 58 | 55 | 52 | 50 | 47 |
| milliseconds | milliseconds | milliseconds | milliseconds | milliseconds | milliseconds | milliseconds | milliseconds | milliseconds | milliseconds |
| 21 | 22 | 23 | 24 | 25 | 26 | 27 | 28 | 29 | 30 |
| minutes | minutes | minutes | minutes | minutes | minutes | minutes | minutes | minutes | minutes |
| 45 | 43 | 41 | 40 | 38 | 37 | 35 | 34 | 33 | 32 |
| milliseconds | milliseconds | milliseconds | milliseconds | milliseconds | milliseconds | milliseconds | milliseconds | milliseconds | milliseconds |

To sum up, it should be noted that the claimed group of inventions enables the differentiation of objective sleep parameters and selection of appropriate amplitude and frequency modes and provides the users with a unique opportunity to decide, on their own, which sleep parameter they want to adjust when using the system in accordance with the method for correcting human sleep parameters disclosed in this patent.

1. A method for correcting human sleep parameters, the method comprising:
generating short (“spike”) electric field pulses of no less than 20 milliseconds to no more than 1000 milliseconds;
using generated pulses near the sleeping person according to at least one of the following algorithms:
improving sleep continuity as measured by the number of transitions from the second stage of the sleep cycle to the first stage and to the waking state;
reducing average sleep latency by gradually changing the duration of pulses over the first 20-30 minutes of sleep;
simplifying awakening process by gradually changing the duration of pulses over the last 30-60 minutes of sleep;
increasing the amount of slow sleep in the second and third stages of the sleep cycle.

2. Method according to claim 1, wherein the pulses are generated by electromagnetic induction.

3. Method according to claim 2, wherein electromagnetic induction is caused by the rate of change in the value of the magnetic field generated by the magnetic field source.

4. Method according to claim 3, wherein the magnetic field source is provided by a flat bifilar inductor coil, while the rate of change in the magnetic field value is controlled by the rate of rise of the leading edge of electric pulses fed to the coil, and wherein such pulses have rectangular shape with a fill factor of less than 5%.

5. The system for correcting human sleep parameters to use the method according to claim 1 and comprising the following:
unit for generating short (“spike”) pulses of the electric field;
memory and control unit to control the said unit for generating pulses;
time control unit to synchronize the generated “spike” pulses with the periods of falling asleep, deep sleep and/or awakening.

6. System according to claim 5, wherein the generation unit produces short (“spike”) pulses by electromagnetic induction.

7. System according to claim 6, wherein the generation unit is designed to change the amplitude of short (“spike”) pulses through the rate of rise and amplitude of the leading edge of electric pulses fed to the time-varying magnetic field shaper.

8. System according to claim 7, wherein the said time-varying magnetic field shaper is made in the form of a flat bifilar inductor coil.

9. System according to claim 8, wherein the inductor coil is designed to receive an amplified electric signal with the specified duration parameters of pulses, which have a rectangular shape with a fill factor of less than 5%.

10. System according to claim 9, wherein the said electric signal with the specified duration parameters and fill factor is generated by the converter.

11. System according to any of claims 5-10, claim 5, wherein the said algorithms are stored in a memory unit synchronized with the said time control unit to ensure that the generated “spike” pulses of electric field with a certain duration are aligned in time with periods of falling asleep, deep sleep and awakening.

12. System according to claim 11, wherein the said control algorithms can be communicated to the said memory unit through a mobile app using the said Bluetooth module, or by a remote server through the said Wi-Fi module.
